(19) United States
(12) Patent Application Publication
(10) Pub. No.: US 2016/0256045 A1
(43) Pub. Date: Sep. 8, 2016

(54) ENDOSCOPE REPROCESSING APPARATUS

(71) Applicant: OLYMPUS CORPORATION, Tokyo (JP)

(72) Inventor: Naoya TAYA, Tokyo (JP)

(21) Appl. No.: 15/152,893

(22) Filed: May 12, 2016

Related U.S. Application Data
(63) Continuation of application No. PCT/JP2015/055903, filed on Feb. 27, 2015.

(30) Foreign Application Priority Data
Jul. 7, 2014 (JP) 2014-139846

(51) Int. Cl. A61B 1/12; A61L 2/24; A61L 2/02; A61B 1/00

(52) U.S. Cl. CPC A61B 1/123; A61B 1/00059; A61L 2/24; A61L 2/02

(57) ABSTRACT

When an opening/closing detecting portion detects that a cover closed, a control portion outputs a signal to an electromagnetic locking switch of a locking mechanism to suppress opening and closing so that the cover enters a state in which the cover cannot be opened by an ordinary operation of a handle or a depression operation of an operation pedal. After that, when an operator operates an operation panel to input start of a process of cleaning, disinfection, etc., the control portion causes a cleaning/disinfecting portion to function and predeterminedly execute the inputted process. Then, when completion of the process of cleaning, disinfection, etc., is detected by a cleaning/disinfection detecting portion, the control portion releases the suppression of opening and closing of the cover by the locking mechanism and displays the completion of the process of cleaning, disinfection, etc., on the operation panel.

### CROSS REFERENCE TO RELATED APPLICATION

This application is a continuation application of PCT/JP2015/055903 filed on Feb. 27, 2015 and claims benefit of Japanese Application No. 2014-139846 filed in Japan on Jul. 7, 2014, the entire contents of which are incorporated herein by this reference.

### BACKGROUND OF THE INVENTION

1. Field of the Invention

The present invention relates to an endoscope reprocessing apparatus which performs a process of rinsing, cleaning, disinfection and the like to reprocess an endoscope.

2. Description of the Related Art

At a time of cleaning and disinfecting a used endoscope using an endoscope reprocessing apparatus, the endoscope is put into a cleaning/disinfecting tank, and various kinds of tubes are connected to connection fittings of the endoscope first. After that, a cover is closed, and a start button is pressed. After that, a cleaning/disinfecting process starts, and, after elapse of a predetermined time period, the cleaning/disinfecting process ends. Lastly, an operator opens the cover and takes out the cleaned and disinfected endoscope.

Here, in the cleaning/disinfecting process described above, it may happen that, after the cover is closed or in a state of being left open after the used endoscope is put into the cleaning/disinfecting tank, the endoscope is left for a long time period without the start button being pressed.

In such a case, there is a possibility that the endoscope which has not been cleaned or disinfected is brought out by another operator different from the operator who put the endoscope into the cleaning/disinfecting tank. Therefore, there has been demand for a function of notifying an operator who brings out the endoscope from the cleaning/disinfecting tank that the endoscope has not been cleaned or disinfected.

In view of such a situation, an endoscope cleaning/disinfecting apparatus of Japanese Patent Application Laid-Open Publication No. 2003-235793 has a function of transmitting information to the effect that an endoscope has been cleaned and disinfected to the endoscope after the cleaning/disinfecting process ends. A microcomputer in a processor in an examination room or an operating room has a function of, when an endoscope is connected to the processor at time of using the endoscope, receiving information about whether the endoscope has been cleaned and disinfected or not, from the endoscope, or issuing a warning if an endoscope which has not been cleaned or disinfected is connected. Thereby, an operator is informed that the endoscope has not been cleaned and disinfected.

### SUMMARY OF THE INVENTION

An endoscope reprocessing apparatus of an aspect of the present invention includes: a tank-type cleaning/disinfecting tank accommodating an endoscope; a cover with which the cleaning/disinfecting tank is freely opened and closed; a locking portion locking the cover in a closed state in which the cover covers the cleaning/disinfecting tank; an opening suppressing portion suppressing the cover from opening a predetermined width or more, in conjunction with the cover entering the closed state; an interface freely accepting external input of a release signal for releasing suppression by the opening suppressing portion; and a releasing portion releasing the suppression by the opening suppressing portion when the release signal is inputted from the interface.

### BRIEF DESCRIPTION OF THE DRAWINGS

FIG. 1 is a perspective view of an endoscope cleaning/disinfecting apparatus according to a first embodiment of the present invention;

FIG. 2 is a schematic explanatory diagram of a configuration of functional portions of the endoscope cleaning/disinfecting apparatus according to the first embodiment of the present invention;

FIG. 3 is an explanatory diagram showing an outline of a control portion of the endoscope cleaning/disinfecting apparatus according to the first embodiment of the present invention;

FIG. 4 is a flowchart showing an endoscope cleaning/disinfecting process of the endoscope cleaning/disinfecting apparatus according to the first embodiment of the present invention;

FIG. 5 is a cross-sectional view in an X direction in FIG. 1 illustrating an outline of a structure of a cleaning/disinfecting tank and a cover when the cover is caused to be in an open state, according to the first embodiment of the present invention;

FIG. 6 is a cross-sectional view of main portions illustrating a configuration of a locking mechanism of the cover, according to the first embodiment of the present invention;

FIG. 7 is a flowchart showing an endoscope cleaning/disinfecting process of an endoscope cleaning/disinfecting apparatus according to a second embodiment of the present invention; and

FIG. 8 is a cross-sectional view of main portions illustrating a configuration of a locking mechanism of a cover according to a third embodiment of the present invention.

### DETAILED DESCRIPTION OF THE PREFERRED EMBODIMENTS

### First Embodiment

A first embodiment of the present invention will be described based on drawings.

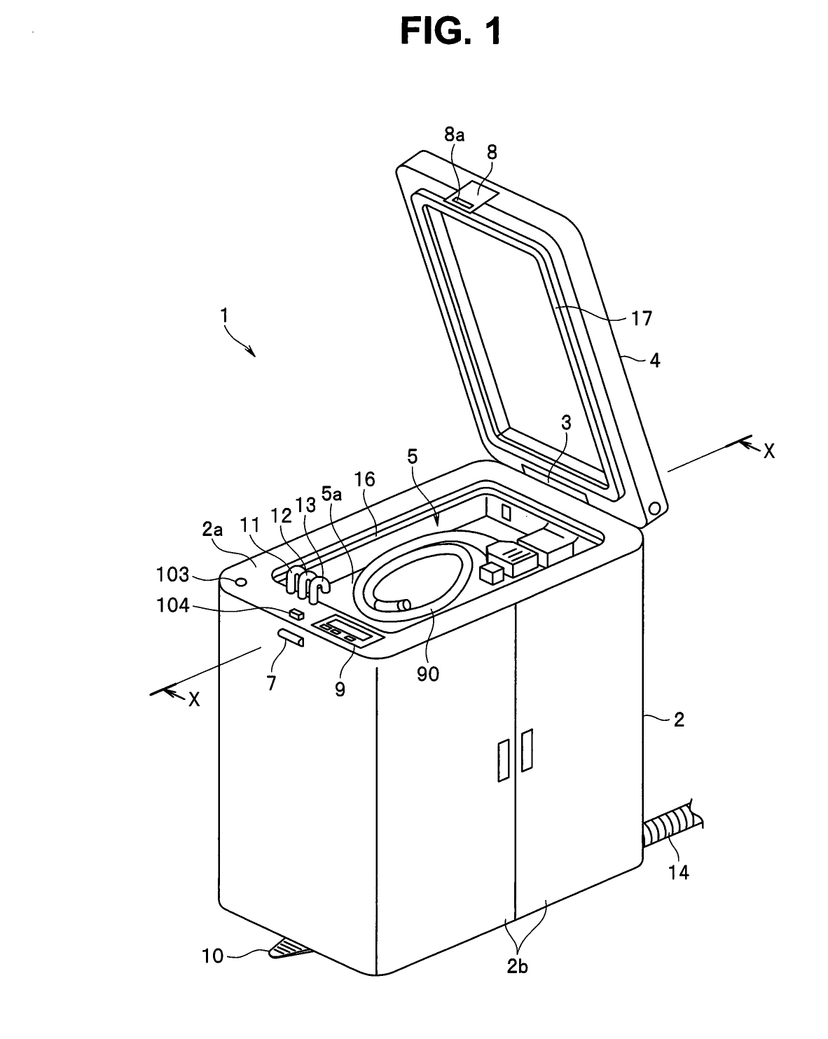

In FIG. 1, reference numeral 1 indicates an endoscope cleaning/disinfecting apparatus as an endoscope reprocessing apparatus. The endoscope cleaning/disinfecting apparatus 1 is configured mainly by a cover 4 which is freely openable and closable via a hinge 3 being provided on a top portion of an apparatus body 2.

On a top face (hereinafter referred to as a “body top face”) 2a of the apparatus body 2, a tank-type cleaning/disinfecting tank (also referred to as a cleaning tank) 5 is arrange which is freely capable of accommodating an endoscope 90 placed on its bottom face 5a for cleaning and disinfection. The cleaning/disinfecting tank 5 is opened and closed by opening and closing the cover 4 described above.

When the endoscope 90 is put into the cleaning/disinfecting tank 5 of the apparatus body 2, and the cover 4 is closed and caused to enter a closed state of substantially covering the cleaning/disinfecting tank 5, a locking portion 7 is engaged with a handle 8 of the cover 4 and causes the cover 4 to be in a completely closed state. For example, a latch structure is conceivable as the locking portion 7.

A lock by the locking portion 7 can be released by releasing portions 8 and 10. As the lock releasing portions 8 and 10, mechanisms which enable the lock to be released by one action are desirable. For example, the handle 8 of the cover 4 which enables an operator to release the lock by performing a rotation operation of the handle 8 or an operation pedal 10 which is provided on a lower portion of the apparatus body 2 and which enables the operator to open the cover 4 by performing a stepping operation are conceivable.

The endoscope reprocessing apparatus of the present invention is provided with an opening suppressing portion 6 which suppresses opening of the cover 4, separately from the locking portion 7.

The opening suppressing portion 6 suppresses the cover 4 from opening a predetermined width or more. The predetermined width stated here may be any width that disables a user to take out an endoscope from the cleaning tank or causes the user to feel it difficult to take out the endoscope. For example, 30 cm to 0 cm is conceivable. Thereby, the user notices that the endoscope included in the cleaning tank has not been reprocessed yet.

In the first embodiment, the opening suppressing portion 6 acts on the locking portion 7. When the cover 4 enters the closed state, and the locking portion 7 locks the cover 4, the opening suppressing portion 6 fixes the lock by the locking portion 7 so that the lock by the locking portion 7 is not released even if the lock releasing portions 8 and 10 are moved.

In order to release suppression by the opening suppressing portion 6, that is, the action on the locking portion 7 stated in the first embodiment, a release signal is inputted to a releasing portion 9 connected to the opening suppressing portion 6.

A way to input the release signal depends on a structure of the releasing portion 9.

For example, if the releasing portion 9 electrically acts on the opening suppressing portion 6, the releasing portion 9 is a touch panel, and a touch on the touch panel is conceivable as an input of the release signal. Otherwise, the releasing portion 9 may be a switch, and pressing the switch is conceivable as an input of the release signal.

For example, if the releasing portion 9 mechanically acts on the opening suppressing portion 6, the releasing portion 9 is a lever, a pedal or a handle, and causing the lever, the pedal or the handle to operate is an input of the release signal.

In FIG. 1, a touch panel 9 is disclosed as the releasing portion 9.

The touch panel may have a display function of displaying an operation state of the endoscope cleaning/disinfecting apparatus 1, an error signal in case of occurrence of an abnormality, and the like, in addition to the function as a releasing portion. Furthermore, the touch panel may have a function as an operation panel as an interface for operating the apparatus body 2. Note that the operation panel 9 may be provided on a side of the apparatus body 2 or on the cover 4.

Furthermore, for example, an RFID (radio frequency identification) reader 103 is provided on the body top face 2a as an endoscope information reading portion for detecting that the endoscope 90 has been put into the cleaning/disinfecting tank 5 by detecting information about the endoscope 90, and the RFID reader 103 is capable of reading the information about the endoscope 90, such as a model name and serial number of the endoscope 90, from an RFID tag or the like which is included in (or provided outside) a connector portion or the like of the endoscope 90 and is not shown. Note that the RFID reader 103 may be provided on a part other than the body top face 2a. Further, the system for reading endoscope information can be realized by another system such as a system using reading by barcode reading, or a method of inputting the endoscope information by an operator.

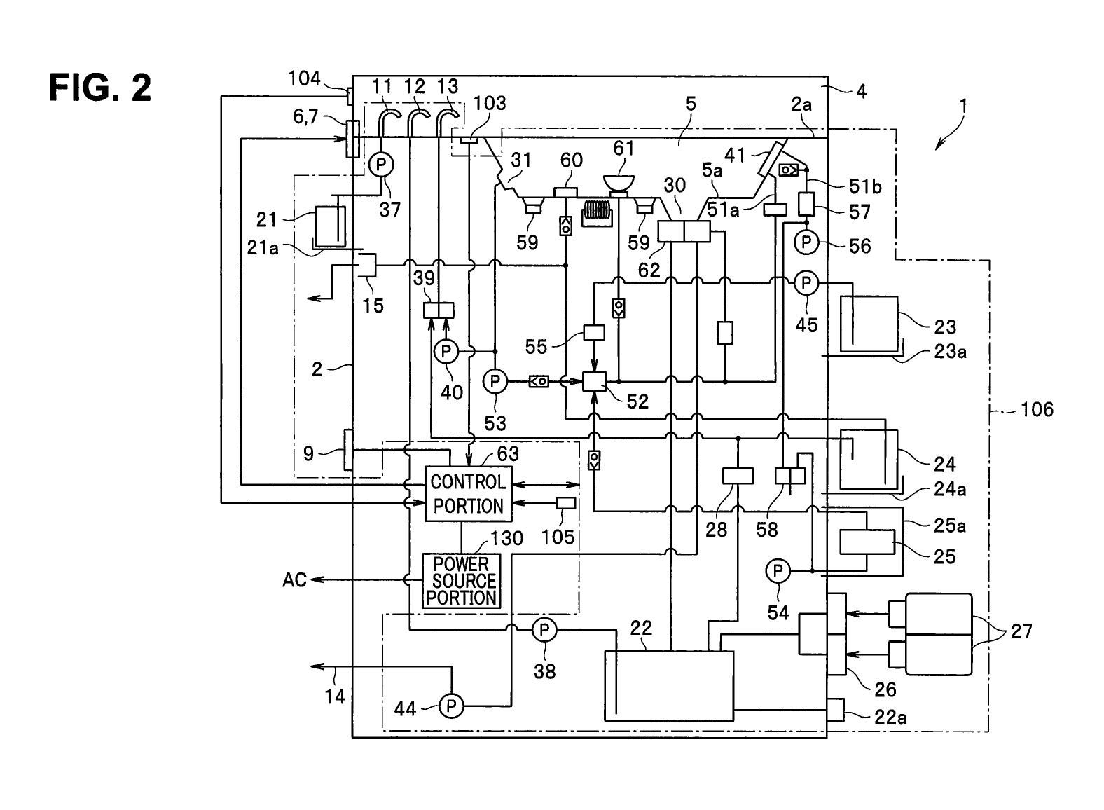

Further, a side door 2b to be described later is arranged on any side of the apparatus body 2. Furthermore, a drain hose 14 for draining cleaning liquid, disinfecting liquid or the like in the cleaning/disinfecting tank 5 is connected to a lower part of the apparatus body 2. Further, the apparatus body 2 is provided with a water supply connector 15 (see FIG. 2) to be described later, which supplies water into the cleaning/disinfecting tank 5.

In the cleaning/disinfecting tank 5, a stepped part 16 in a stepped shape is formed on substantially an entire periphery of an edge portion of an opening portion on the body top face 2a. A packing member 17 for water-tightly sealing the cover 4 and the cleaning/disinfecting tank 5 when the cover 4 is closed and for keeping a cushioning property at time of closing the cover 4 is provided along the shape of the stepped part 16, on a part facing the stepped part 16 on an internal face of the cover 4. Note that a central part of the cover 4 is formed with transparent resin, glass material or the like so that an inside of the cleaning/disinfecting tank 5 can be seen when the cover 4 is closed.

Further, on a part facing a bottom face 4a of the cover 4, between the handle 8 and the packing member 17, an opening/closing detecting portion 104 as a closed state detecting portion which detects that the cover 4 has entered the closed state by detecting contact with the bottom face 4a of the cover 4 is provided.

Furthermore, a cleaner nozzle 11, a disinfecting liquid nozzle 12, a water supply/circulation nozzle 13 to be described later are arranged on the body top face 2a. The nozzles 11, 12 and 13 constitute a cleaning/disinfecting portion 106 as a sanitizing portion which performs sanitizing such as rinsing, cleaning, and disinfecting processes of the endoscope 90. The cleaning/disinfecting portion 106 will be described below based on FIG. 2.

As shown in FIG. 2, a cleaner tank 21 for storing liquid cleaner, a disinfecting liquid tank 22 for storing disinfecting liquid diluted to a predetermined concentration, an alcohol tank 23 for storing alcohol, a water filter 24 for filtering tap water supplied from a tap, and an air filter 25 are arranged inside the apparatus body 2. The disinfecting liquid tank 22 is fixed in the apparatus body 2. Note that reference numeral 22a indicates a disinfecting liquid drain port, which is usually closed.

Further, the cleaner tank 21, the alcohol tank 23, the water filter 24, the air filter 25 are placed on trays 21a, and 23a to 25a, respectively. Further, each of the trays 21a, and 23a to 25a can be laterally and freely drawn by opening the side door 2b (see FIG. 1) of the apparatus body 2 so that liquid can be predeterminedly replenished, or parts can be freely exchanged.

In the present embodiment, at time of replenishing the disinfecting liquid tank 22 with disinfecting liquid, the replenishment is performed, for example, by opening the side door 2b of the apparatus body 2 and connecting a disinfecting liquid bottle 27 filled with the disinfecting liquid to a bottle connector 26 fixed inside the apparatus.

Further, at that time, tap water filtered by the water filter 24 is supplied to the disinfecting liquid tank 22 via a dilution valve 28. Therefore, disinfecting liquid diluted to a predetermined concentration is stored in the disinfecting liquid tank 22. Note that FIG. 2 shows a state in which each of the trays 21a, and 23a to 25a has been drawn out.

The cleaning/disinfecting tank 5 is capable of freely accommodating the endoscope 90 as described above, and a drain port 30 is provided in the bottom face 5a. Furthermore, a circulation port 31 is provided in one side of on an outer circumferential wall face of the cleaning/disinfecting tank 5. Further, the cleaner nozzle 11, the disinfecting liquid nozzle 12, the water supply/circulation nozzle 13 are arranged at a corner portion of the body top face 2a, on a side where the water supply connector 15 is arranged.

The cleaner nozzle 11 communicates with the cleaner tank 21 via a cleaner pump 37, and the disinfecting liquid nozzle 12 communicates with a disinfecting liquid tank 22 via a chemical pump 38. Further, the water supply/circulation nozzle 13 can be selectively and freely connected to the water filter 24 or a flowing liquid pump 40 via a three-way valve 39.

In a state that the water supply/circulation nozzle 13 is connected to the water filter 24 side via the three-way valve 39, tap water filtered by the water filter 24 is discharged from the water supply/circulation nozzle 13 to the cleaning/disinfecting tank 5.

On the other hand, in a state that the water supply/circulation nozzle 13 is connected to the flowing liquid pump 40 via the three-way valve 39, cleaning water or disinfecting liquid taken in from the circulation port 31 which has been stored in the cleaning/disinfecting tank 5 is again discharged into the cleaning/disinfecting tank 5 and circulated.

Note that a high-pressure nozzle is connected between the water supply/circulation nozzle 13 and the three-way valve 39 via a high-pressure pump though it is not shown, and liquid similar to that of the water supply/circulation nozzle 13 (tap water or cleaning water) is also spouted from the high-pressure nozzle into the cleaning/disinfecting tank 5 at a high pressure.

A water current is generated in the cleaning/disinfecting tank 5 by the liquids discharged from the high-pressure nozzle and the water supply/circulation nozzle 13, and, by the water current, an outer surface of the endoscope 90 is cleaned in the cleaning process, and cleaning liquid or disinfecting liquid is washed away in the rinsing process.

On the other side of the outer circumferential wall face of the cleaning/disinfecting tank 5, a connector receiving portion 41 to be connected to a conduit connector portion provided on the endoscope 90, which is not shown, is provided. One cleaning/disinfecting tube 51a is branch-connected to the connector receiving portion 41, and the cleaning/disinfecting tube 51a communicates with a discharge port of a channel block 52 configured with a four-way valve.

Further, the circulation port 31, the alcohol tank 23 and a compressor 54 communicate with three branched inflow ports of the channel block 52, respectively. Further, a channel pump 53 for sucking fluid (tap water, cleaning water or disinfecting liquid) from the circulation port 31 is interposed between the circulation port 31 and the channel block 52.

Furthermore, an alcohol valve 55 for opening and closing a flow path and an alcohol pump 45 for sucking alcohol stored in the alcohol tank 23 are interposed between the alcohol tank 23 and the channel block 52. Further, the air filter 25 is interposed between the compressor 54 and the channel block 52.

By causing the channel block 52 to perform a switching operation to selectively communicate each inflow port with the discharge port, liquid (tap water, cleaning water or disinfecting liquid) stored in the cleaning/disinfecting tank 5, alcohol stored in the alcohol tank 23 or air from the compressor 54 is supplied to the endoscope 90.

Further, a water leak detecting pump 56 is connected to the connector receiving portion 41 via a water leak detecting tube 51b, and a cutoff valve 57 is provided for the water leak detecting tube 51b. At time of detecting whether or not there is a small hole, a crack or the like on the outer surface of the endoscope 90, the cutoff valve 57 is opened first, and then air from the water leak detecting pump 56 is supplied to an inside of the endoscope 90 to predeterminedly increase internal pressure.

After that, the cutoff valve 57 is closed to keep the internal pressure of the endoscope 90. Then, it is checked whether or not there is a small hole, a crack or the like on the outer surface of the endoscope 90, from change in the internal pressure of the endoscope 90 during the meantime. Note that reference numeral 58 indicates an exhaust valve, and air from the water leak detecting pump 56 and the compressor 54 can be discharged to an outside by opening the exhaust valve 58.

Further, an ultrasound transducer 59, a suction tube disinfecting connector 60, a cleaning case 61 and the like are predeterminedly arranged in the cleaning/disinfecting tank 5, and, furthermore, a switching valve 62 is arranged at the drain port 30. The ultrasound transducer 59 vibrates cleaning water or tap water stored in the cleaning/disinfecting tank 5 to perform ultrasound cleaning or rinsing of the outer surface of the endoscope 90.

The disinfecting liquid nozzle 12 is connected to the suction tube disinfecting connector 60 via a hose and the like to supply disinfecting liquid to a water supply tube communicating with the water filter 24 and disinfect the water supply tube. Further, removable parts provided for the endoscope 90, such as buttons of respective scope switches of the endoscope 90, are put into the cleaning case 61 to clean and disinfect the parts together with the endoscope 90.

Furthermore, the switching valve 62 arranged at the drain port 30 is for switching between drain paths at time of drainage. When tap water or cleaning water is stored in the cleaning/disinfecting tank 5, the switching valve 62 causes the drain port 30 to communicate with the drain hose 14 side and drives a drain pump 44 to force the water to be drained.

On the other hand, when disinfecting liquid is stored in the cleaning/disinfecting tank 5, the switching valve 62 causes the drain port 30 to communicate with the disinfecting liquid tank 22 side to collect disinfecting liquid used for disinfection into the disinfecting liquid tank 22. Therefore, the disinfecting liquid is repeatedly used.

A switching operation among the respective valves 39, 52, 55 and 62 and operations of the various pumps 37, 38, 40 53 and 56 are controlled by a control portion 63 included in the apparatus body 2.

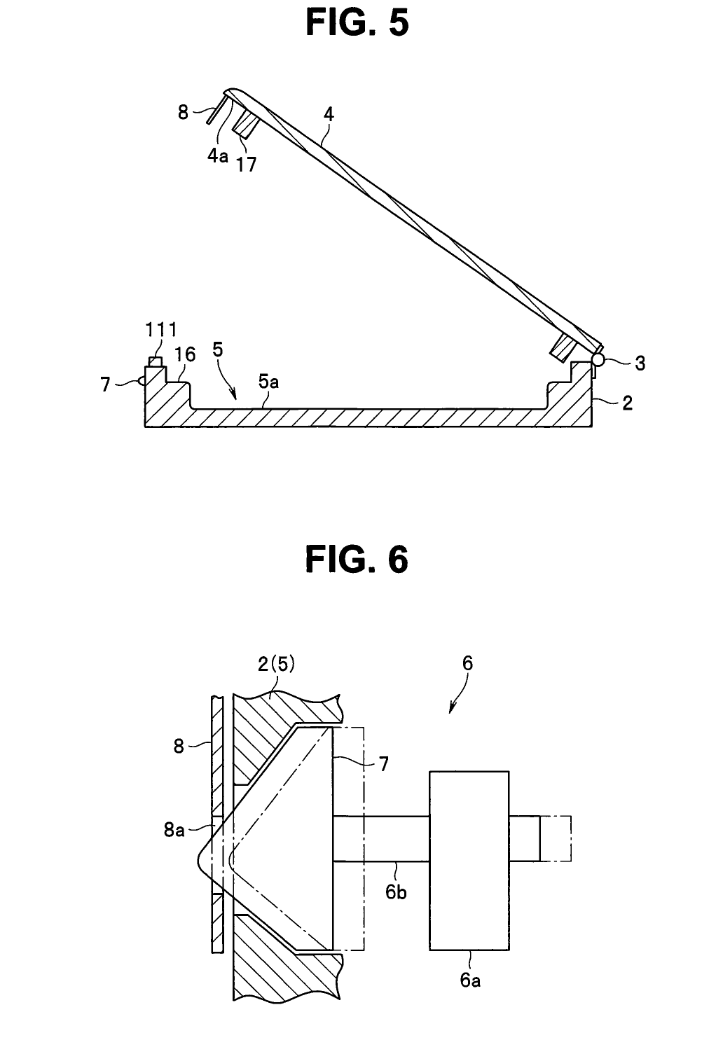

Next, a locking mechanism 6 will be described based on FIGS. 5 and 6.

FIG. 5 shows an outline of a structure of the cleaning/disinfecting tank 5 part on the top part of the apparatus body 2, and the cover 4 when the cover 4 is in an opened state. The cover 4 which is freely opened and closed provided on the top part of the apparatus body 2 via the hinge 3 can be opened at a predetermined large angle. When the cover 4 is opened at an angle not less than a predetermined angle, a weak urging force acts in an opening direction to keep the opened state, so that the endoscope 90 can be easily and freely put into the cleaning/disinfecting tank 5. The urging force is adapted to, when the cover 4 is closed to an angle smaller than the predetermined angle, act weakly in the closing direction to keep the closed state of the cover 4.

Further, in a state that the cover 4 is opened, the latch 7 provided to correspond to the handle 8 of the cover 4 is in a state that its distal end projects toward an outer side. That is, as shown in FIG. 6, in the locking mechanism 6 provided for the cleaning/disinfecting tank 5 part on the top part of the apparatus body 2, the latch 7 is continuously pressed and urged by a rod 6b projecting from an electromagnetic locking switch 6a toward the outer side (the handle 8 side of the cover 4), and a distal end of the latch 7 projects.

As shown in FIG. 6, at time of trying to close the cover 4 from the state that the cover 4 is opened, the latch 7 is thrust to an inner side against an urging force, by movement of the handle 8 of the cover 4. When a hole portion 8a formed on a distal end side of the handle 8 reaches a substantially distal end of the latch 7, the latch 7 again projects toward the outer side from the hole portion 8a of the handle 8.

When the control portion 63 detects that the cover 4 has entered the closed state, by a signal from the opening/closing detecting portion 104, the electromagnetic locking switch 6a functions (is turned on), and the rod 6b is fixed. Thereby, the handle 8 is locked with the distal end of the latch 7 being fixed in the state of projecting from the hole portion 8a of the handle 8 toward the outer side, so that the state that the cover 4 is closed is certainly kept. The locked state of the cover 4 by the locking mechanism 6 is released by the control portion 63 when a release (signal) is inputted (the locked state is turned off) from the operation panel 9 by an operator or the like. Thus, the control portion 63 has a function as a releasing portion.

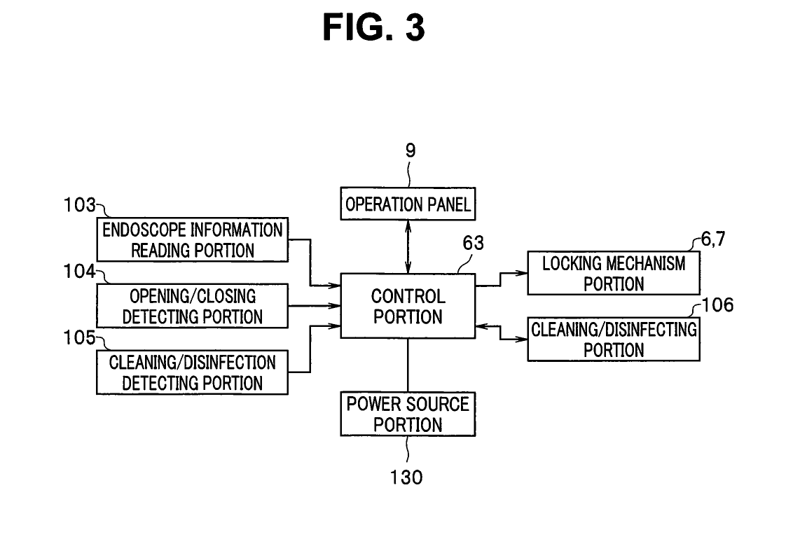

As shown in FIG. 3, the control portion 63 of the endoscope cleaning/disinfecting apparatus 1 described above is configured mainly by locking mechanism portions 6 and 7, the operation panel 9, the endoscope information reading portion (RFID reader) 103, the opening/closing detecting portion 104, a cleaning/disinfection detecting portion 105, the cleaning/disinfecting portion 106 and a power source portion 130 being connected to the control portion 63. Here, the cleaning/disinfection detecting portion 105 detects an operation state (a driven state or a progress state) of the cleaning/disinfecting portion 106 and is configured, for example, with a water level sensor, a pressure sensor, a temperature sensor and other sensors.

When it is detected by the opening/closing detecting portion 104 that the cover has been closed, the control portion 63 outputs a signal to the electromagnetic locking switch 6a of the locking mechanism 6 to lock the cover 4 to be in a state in which the cover 4 cannot be opened by an ordinary operation of the handle 8 or a depression operation of the operation pedal 10. After that, when an operator operates the operation panel 9 to input start of a process of cleaning, disinfection and the like, the control portion 63 causes the cleaning/disinfecting portion 106 to function and predeterminedly execute the inputted process. Then, when completion of the process of cleaning, disinfection and the like is detected by the cleaning/disinfection detecting portion 105, the control portion 63 releases the lock by the locking mechanism 6 and displays the completion of the process of cleaning, disinfection and the like on the operation panel 9.

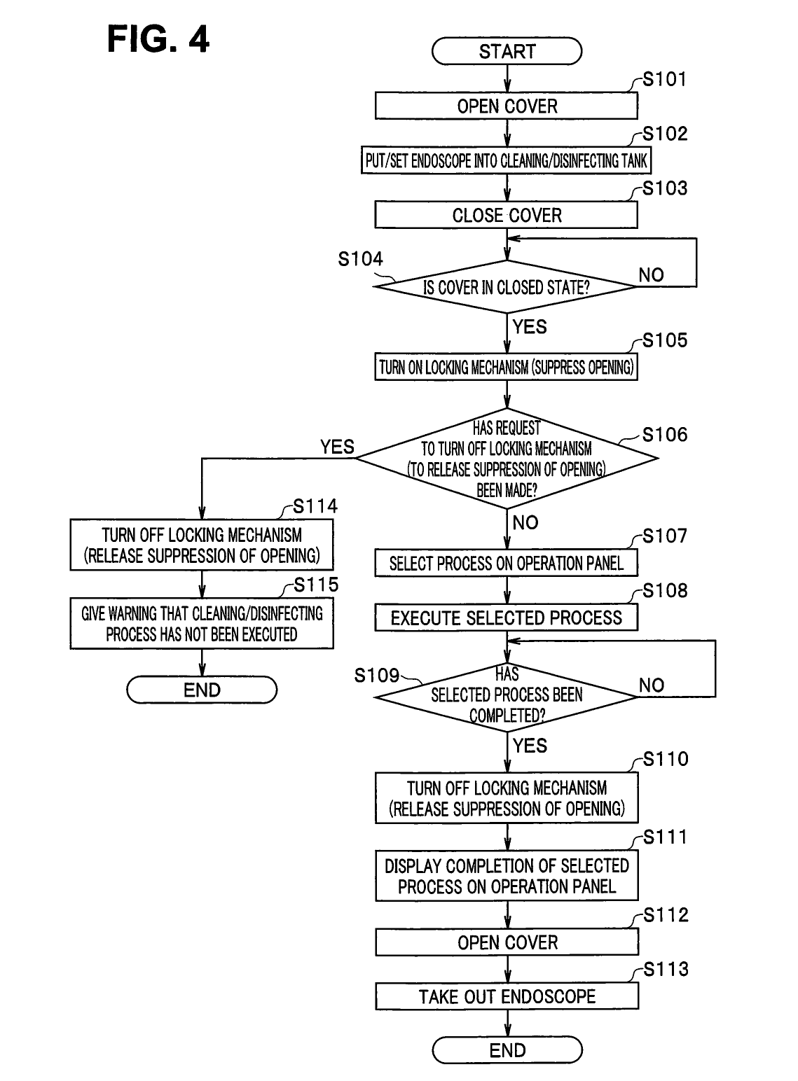

The cleaning/disinfecting process for the endoscope 90 executed by the control portion 63 of the endoscope cleaning/disinfecting apparatus 1 will be described below according to a flowchart shown in FIG. 4.

First, at step (hereinafter abbreviated as “S”) 101, an operator opens the cover 4 of the endoscope cleaning/disinfecting apparatus 1, for example, by operating the handle 8 or performing a depression operation of the operation pedal 10.

Next, the process proceeds to S102, and the operator puts and sets the endoscope 90 to be cleaned and disinfected, into the cleaning/disinfecting tank 5.

Then, the process proceeds to S103, and the operator closes the cover 4.

Then, when the process proceeds to S104, the control portion 63 judges whether or not the cover 4 has entered the closed state, based on a signal from the opening/closing detecting portion 104. If judging that the cover 4 has entered the closed state, the control portion 63 proceeds to S105, and causes the electromagnetic locking switch 6a of the locking mechanism 6 to function (to be turned on) to cause the cover 4 to enter an opening suppressed state. Note that it is also possible to, after elapse of a predetermined time period after it is judged at S104 that the cover 4 has entered the closed state, proceed to S105 and cause the electromagnetic locking switch 6a of the locking mechanism 6 to function (to be turned on) to cause the cover 4 to enter the opening suppressed state.

Next, when the process proceeds to S106, it is judged whether or not the operator has made a request to release the suppression of opening by the locking mechanism 6 from the operation panel 9.

If the operator has not made the request to release the locking mechanism 6, the process proceeds from S106 to S107, and the process of cleaning, disinfection and the like on the operation panel 9 is selected.

Then, the process proceeds to S108, and the control portion 63 causes the cleaning/disinfecting portion 106 to function and predeterminedly execute the selected (inputted) process.

Next, the process proceeds to S109, and the control portion 63 judges whether the process of cleaning, disinfection and the like has been completed or not by the cleaning/disinfection detecting portion 105. If the process has been completed, the process proceeds to S110, and the lock by the locking mechanism 6 is turned off (the suppression of opening is released).

Next, the process proceeds to S111, and the control portion 63 displays completion of the process of cleaning, disinfection and the like on the operation panel 9.

After that, the process proceeds to S112, and the operator opens the cover 4, for example, by operating the handle 8 or performing a depression operation of the operation pedal 10. Then, the process proceeds to S113, and the operator takes out the endoscope 90 from the cleaning/disinfecting tank 5 and ends the cleaning/disinfecting process for the endoscope 90.

On the other hand, if the operator has made the request to turn off the locking mechanism 6 (release the suppression of opening) by operating the operation panel 9 for some reason (for example, in a case of changing a setting position of the accommodated endoscope to a more favorable position), the process proceeds to S114, and the control portion 63 turns off the lock by the locking mechanism 6 (releases the suppression of opening).

Then, the process proceeds to S115, and it is displayed on the operation panel 9 that the cleaning/disinfecting process has not been executed for the endoscope 90 in the cleaning/disinfecting tank 5 to give a warning. Note that, for the warning, an auditory warning such as a voice and a buzzer may be simultaneously given in addition to the visual display by the operation panel 9.

Thus, according to the first embodiment of the present invention, when it is detected by the opening/closing detecting portion 104 that the cover 4 has been closed, a signal is outputted to the locking mechanism 6 to lock the cover 4 in the state that the cover 4 cannot be opened by an ordinary operation of the handle 8 or a depression operation of the operation pedal 10. After that, when the operator operates the operation panel 9 to input start of the process of cleaning, disinfection and the like, the control portion 63 causes the cleaning/disinfecting portion 106 to function to predeterminedly execute the inputted process. Then, when completion of the process of cleaning, disinfection and the like is detected by the cleaning/disinfection detecting portion 105, the control portion 63 releases the lock by the locking mechanism 6 and displays the completion of the process of cleaning, disinfection and the like on the operation panel 9.

Thus, when the cover 4 is closed, the locking mechanism 6 functions. Therefore, it is possible to, when the endoscope 90 is put into the cleaning/disinfecting tank 5 to clean and disinfect the endoscope 90, prevent the endoscope 90 which has not been subjected to reprocessing such as the cleaning/disinfecting process from being brought out by an operator even if the operator is different from a person who has put the endoscope 90 into the cleaning/disinfecting tank 5 or even if the person who has put the endoscope 90 into the cleaning/disinfecting tank 5 fails to operate the operation panel 9 or fails to make a confirmation, because the cover 4 is locked by the locking mechanism 6 so as not to be opened.

Further, if the locked state of the cover 4 is released for some reason, a warning is given by the operation panel 9. Therefore, an operator who tries to open the cover 4 can certainly recognize that the cleaning/disinfecting process and the like have not been executed for the endoscope 90 put in the cleaning/disinfecting tank 5.

### Second Embodiment

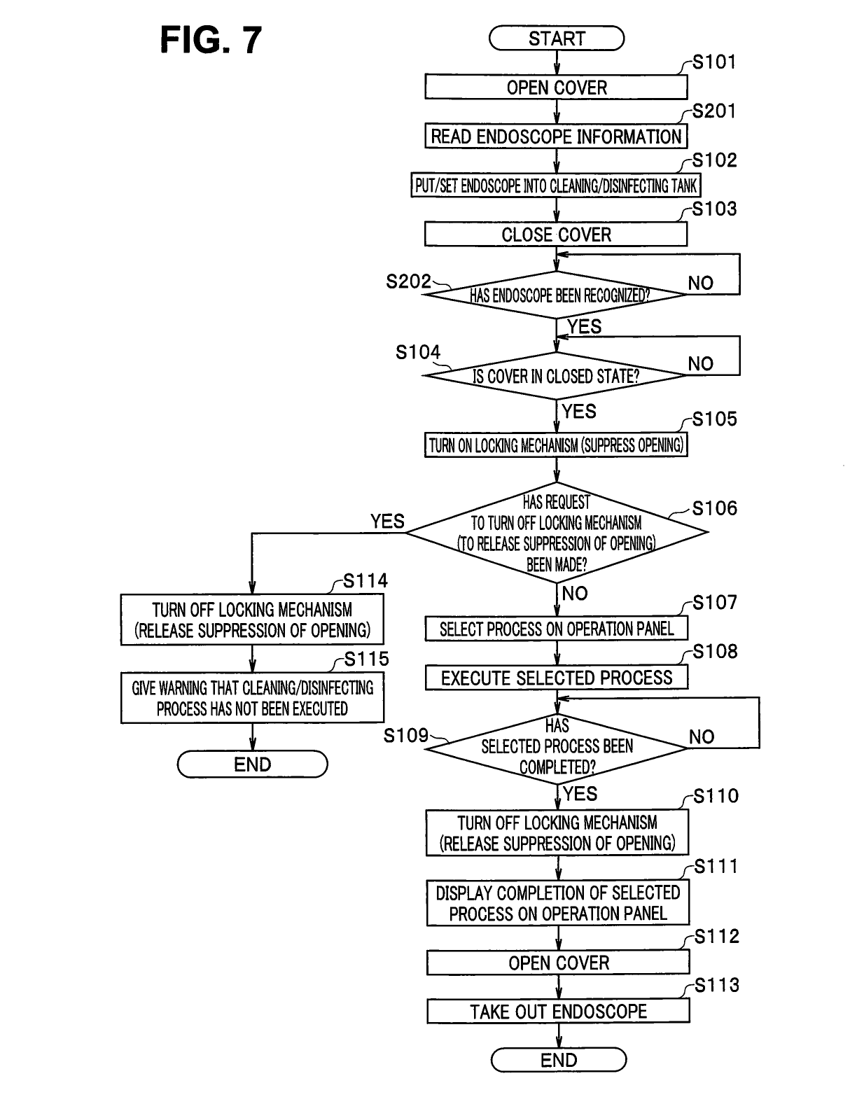

Next, a second embodiment of the present invention will be described based on FIG. 7.

Note that the second embodiment of the present invention is different from the first embodiment in that the second embodiment is configured so that the cover 4 is not locked when the cover 4 is closed without the endoscope 90 being accommodated. Since other components, operation and advantageous effects are similar to those of the first embodiment, the same components are given the same reference numerals, and description of the components will be omitted.

That is, at S101 in a flowchart of a cleaning/disinfecting process for the endoscope 90 in FIG. 7, an operator opens the cover 4 of the endoscope cleaning/disinfecting apparatus 1, for example, by operating the handle 8 or performing a depression operation of the operation pedal 10.

Next, the process proceeds to S201, and information about the endoscope 90 is read in by the RFID reader 103.

Next, the process proceeds to S102, and the operator puts and sets the endoscope 90 to be cleaned and disinfected, into the cleaning/disinfecting tank 5.

Then, the process proceeds to S103, and the operator closes the cover 4.

Next, the process proceeds to S202, and the control portion 63 judges whether or not the endoscope 90 has been recognized by the RFID reader 103. Note that the endoscope information reading process of S201 is only required to be performed before the process of S202. For example, the endoscope information may be read in at the same time the endoscope is put in and set at S102 described above.

If the endoscope 90 has been recognized as a result of the judgment at S202, the process proceeds to S104, and the control portion 63 judges whether or not the cover 4 has entered the closed state, based on a signal from the opening/closing detecting portion 104. If judging that the cover 4 has entered the closed state, the control portion 63 proceeds to S105, and causes the electromagnetic locking switch 6a of the locking mechanism 6 to function (to be turned on) to cause the cover 4 to enter the opening suppressed state. Note that it is also possible to, after elapse of a predetermined time period after it is judged at S104 that the cover 4 has entered the closed state, proceed to S105 and cause the electromagnetic locking switch 6a of the locking mechanism 6 to function (to be turned on) to cause the cover 4 to enter the opening suppressed state.

After the cover 4 is caused to enter the opening suppressed state at S105, the process proceeds to S106 and subsequent steps, and processes similar to the processes described in the first embodiment described above are performed.

On the other hand, if the endoscope 90 has not been recognized as a result of the judgment at S202 described above, waiting occurs until the endoscope 90 is recognized so that the cover 4 is not locked even if the cover 4 is closed.

Thus, according to the second embodiment of the present invention, if the opening/closing detecting portion 104 detects the closed state of the cover 4 after the RFID reader 103 reads endoscope information, the cover 4 is locked by the locking mechanism 6. Therefore, in addition to the advantageous effect of the first embodiment described above, an advantageous effect is also obtained that, in a state that the endoscope 90 is not accommodated, the cover 4 is not locked even if the cover 4 is closed, and, therefore, it is prevented from happening that the cover 4 is closed and locked by mistake though the endoscope 90 is not accommodated.

### Third Embodiment

Next, a third embodiment of the present invention will be described based on

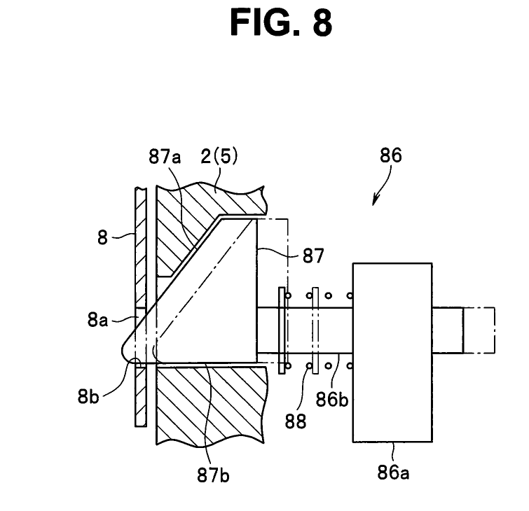

FIG. 8.

Note that, in the third embodiment of the present invention, a shape (configuration) of a locking mechanism is changed from that of the first embodiment described above so that the locking mechanism 6 is caused to detect the closed state when the cover 4 is closed by an operator (S104) and perform the function of locking the cover 4 (S105) itself. Since other components, operation and advantageous effects are similar to those of the first embodiment, the same components are given the same reference numerals and description of the components will be omitted.

That is, as shown in FIG. 8, a locking mechanism 86 of the present third embodiment is provided on the cleaning/disinfecting tank 5 part on the top part of the apparatus body 2, and a latch 87 integrally formed at a distal end of a rod 86b is continuously pressed and urged toward the outer side (the handle 8 side of the cover 4) by a spring 88, so that a distal end of a latch 87 projects.

A face 87a facing the handle 8 of the latch 87 is formed so that it is inclined so as to be gradually separated from the handle 8, starting from the hole portion 8a of the handle 8. Further, a face of the latch 87 on a side where the handle 8 is locked (a bottom face in FIG. 8) 87b is formed at an angle substantially orthogonal to an engagement face 8b, which is a lower face of the hole portion 8a of the handle 8, so as to be substantially orthogonal to a movement locus of the hole portion 8a of the handle 8 accompanying opening or closing of the cover 4.

Further, the rod 86b is provided with an electromagnetic locking switch 86a. When a signal from the control portion 63 is inputted, the rod 86b can be caused to freely move inside the apparatus body 2 together with the latch 87.

In the locking mechanism 86 configured as described above, at time of going to close the cover 4 from the state that the cover 4 is opened, a lower edge of the handle 8 presses the facing face 87a of the latch 87 by movement of the handle 8 of the cover 4, and the latch 87 is thrust to the inner side of the apparatus body 2 against an urging force of the spring 88.

Then, when the cover 4 enters a substantially closed state, and the hole portion 8a formed on the distal end side of the handle 8 reaches a substantially distal end of the latch 87, the latch 87 is caused to project from the hole portion 8a of the handle 8 toward the outer side by the urging force of the spring 88.

Thereby, the engagement face 8b of the hole portion 8a of the handle 8 is engaged with the bottom face 87b of the latch 87, and a locked state is obtained in which the cover 4 is never opened, for example, even if the operator operates the handle 8 or performs a depression operation of the operation pedal 10. The locked state of the cover 4 by the locking mechanism 86 is released by the control portion 63 moving the rod 86b to the inner side of the apparatus body 2 together with the latch 87 when the operator or the like inputs a release (signal) from the operation panel 9.

Thus, according to the present third embodiment, a configuration is made which enables the locking mechanism 86 itself to detect the closed state when the cover 4 is closed by the operator and perform the function of locking the cover 4. Therefore, in addition to the advantageous effect of the first embodiment described above, it is possible to use one device as the locking mechanism portions 6 and 7 and the opening/closing detecting portion 104 involved in locking of the cover 4 and perform respective processes collectively, and, therefore, it is possible to make the cleaning/disinfecting process more simple.

### Fourth Embodiment

The first and second embodiments disclose examples in which an opening suppressing portion acts on a locking portion. The fourth embodiment discloses an example in which an opening control portion acts on the hinge 3 for opening and closing the cover 4. As for parts to which the first or second embodiment is applicable, however, description of the parts will be omitted.

When the hinge 3 is configured with use of simple engagement, an opening control portion is a member for preventing rotation of the hinge 3. In this case, at the same time when the cover 4 is caused to enter the closed state by the locking portion 7, or after elapse of a predetermined time period after the cover 4 is caused to enter the closed state by the locking portion 7, the opening control portion acts on the hinge 3 to prevent rotation.

If the hinge 3 has an opening/closing mechanism provided with an electric actuator as indicated by reference numeral 10 in International Publication No. WO2014/045718, the opening control portion is electrically connected to the electric actuator. In this case, at same time when the cover 4 is caused to enter the closed state by the locking portion 7, or after elapse of a predetermined time period after the cover 4 is caused to enter the closed state by the locking portion 7, the opening control portion acts on the electric actuator to cause the electric actuator to continue to generate a force of pressing the cover portion to the cleaning tank.

What is claimed is:

1. An endoscope reprocessing apparatus comprising:
a tank-type cleaning/disinfecting tank accommodating an endoscope;
a cover with which the cleaning/disinfecting tank is freely opened and closed;
a locking portion locking the cover in a closed state in which the cover covers the cleaning/disinfecting tank;
an opening suppressing portion suppressing the cover from opening a predetermined width or more, in conjunction with the cover entering the closed state;
an interface freely accepting external input of a release signal for releasing suppression by the opening suppressing portion; and
a releasing portion releasing the suppression by the opening suppressing portion when the release signal is inputted from the interface.

2. The endoscope reprocessing apparatus according to claim 1, comprising a sanitizing portion sanitizing the endoscope in the cleaning/disinfecting tank and a sanitation state detecting portion detecting an operation state of the sanitizing portion; wherein
the releasing portion releases the suppression by the opening suppressing portion when the sanitation state detecting portion detects an end of operation of the sanitizing portion.

3. The endoscope reprocessing apparatus according to claim 1, comprising a closed state detecting portion detecting the cover changing from an opened state to the closed state, and an endoscope information reading portion reading endoscope information; wherein
the opening suppressing portion performs the suppression when the closed state detecting portion detects the closed state of the cover after the endoscope information reading portion reads the endoscope information.

4. The endoscope reprocessing apparatus according to claim 1, wherein the opening suppressing portion is provided for the locking portion and locks the locking portion to suppress the cover from opening the predetermined width or more.
